(19) United States
(12) Patent Application Publication
(10) Pub. No.: US 2023/0142864 A1
(43) Pub. Date: May 11, 2023

(54) Estimation of Admixture Generation

(71) Applicant: 23andMe, Inc., Sunnyvale, CA (US)

(72) Inventors: Katarzyna Bryc, Redwood City, CA (US); Eric Yves Jean-Marc Durand, Brunstatt-Didenheim (FR); Joanna Louise Mountain, Menlo Park, CA (US); Robin Patrick Smith, Mountain View, CA (US); Peilun Shan, Redmond, WA (US); Bradley Kittredge, San Francisco, CA (US)

(21) Appl. No.: 18/096,868

(22) Filed: Jan. 13, 2023

Related U.S. Application Data
(63) Continuation of application No. 16/946,829, filed on Jul. 8, 2020.
Application No. 16/946,829 is a continuation of application No. 14/924,562, filed on Oct. 27, 2015, now abandoned.
(60) Provisional application No. 62/072,338, filed on Oct. 29, 2014.

(51) Int. Cl. G16B 40/00; G16B 10/00

(52) U.S. Cl. CPC G16B 40/00; G16B 10/00

(57) ABSTRACT

Admixture generation determination includes: obtaining ancestry assignment information associated with an individual's genotype data, the ancestry assignment information at least indicating that a portion of the individual's genotype data is deemed to be associated with a specific ancestry; determining the individual's genetic ancestry summary data corresponding to the specific ancestry; estimating an admixture generation associated with the specific ancestry, the admixture generation indicating a most recent generation or a most recent generation range from which the individual has at least one non-admixed ancestor of the specific ancestry, the estimation including a maximum likelihood determination based at least in part on the individual's genetic ancestry summary data and a recombination model; and outputting the estimated admixture generation.

### CROSS-REFERENCE TO RELATED APPLICATIONS

This application is a continuation of and claims priority to U.S. patent application Ser. No. 16/946,829, filed Jul. 8, 2020, which is hereby incorporated by reference in its entirety.

U.S. patent application Ser. No. 16/946,829 is a continuation of and claims priority to U.S. patent application Ser. No. 14/924,562, filed Oct. 27, 2015, which is hereby incorporated by reference in its entirety.

U.S. patent application Ser. No. 14/924,562 is a continuation of and claims priority to U.S. provisional patent application No. 62/072,338, filed Oct. 29, 2014, which is hereby incorporated by reference in its entirety.

### BACKGROUND OF THE INVENTION

Many present-day people have ancestors that came from different places of the world. Traditional genealogical and ancestry studies rely on surnames and historical records (e.g., registries of births and marriages, etc.) to determine people's ancestries. These traditional techniques can be very limited because ancestry records, especially records dating back many generations, are often incomplete.

In recent years, techniques have been developed using people's genetic information to trace ancestries. In the context of genealogical studies based on genetic information, “genetic admixture” occurs when individuals from two or more separate populations begin producing offspring, and the resulting descendants are referred to as “admixed.” Many existing genetics-based analytics tools, however, are geared towards geneticists conducting population-based studies rather than individuals interested to learn about their own ancestries.

Certain genetics-based ancestry estimation tools are capable of analyzing an admixed individual's genome, comparing the individual's genome with reference models corresponding to various geographical regions, and determining percentages of the individual's genome that are inherited from ancestors from specific geographical regions. For example, certain analysis tools may indicate that an individual has 70%, 25%, 3.3%, and 1.7% of his genome attributed to ancestors that are West African, Italian, Scandinavian, and Native American, respectively. It is likely that the individual has some knowledge about ancestries associated with the larger percentages of the genome because they are typically inherited from recent ancestors such as parents or grandparents. It can be difficult to trace ancestries associated with the smaller percentages as they may go back many generations. Given the ancestry proportion estimates, an individual often wishes to know how many generations ago there was an un-admixed ancestor (also referred to as a full-blooded ancestor) born by parents from a specific geographical region.

### BRIEF DESCRIPTION OF THE DRAWINGS

Various embodiments of the invention are disclosed in the following detailed description and the accompanying drawings.

FIG. 1 is a functional diagram illustrating a programmed computer system for admixture generation estimation in accordance with some embodiments.

FIG. 2 is a block diagram illustrating an embodiment of a system for admixture generation estimation.

FIG. 3 is a flowchart illustrating an embodiment of a process for admixture generation estimation.

FIG. 4 is a diagram illustrating an example of a recombination model.

FIG. 5 is a flowchart illustrating an embodiment of a process for estimating admixture generation using a model such as the model represented using Table 1.

FIG. 6 is a user interface diagram illustrating an example screen displaying admixture generation information.

FIG. 7 is a user interface diagram illustrating another example screen displaying admixture generation information.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

An admixture generation estimation technique is disclosed. For an individual associated with a specific ancestry (e.g., a geographical region), an admixture generation refers to the most recent generation or a most recent generation range from which the individual has at least one non-admixed (full-blooded) ancestor of the specific ancestry.

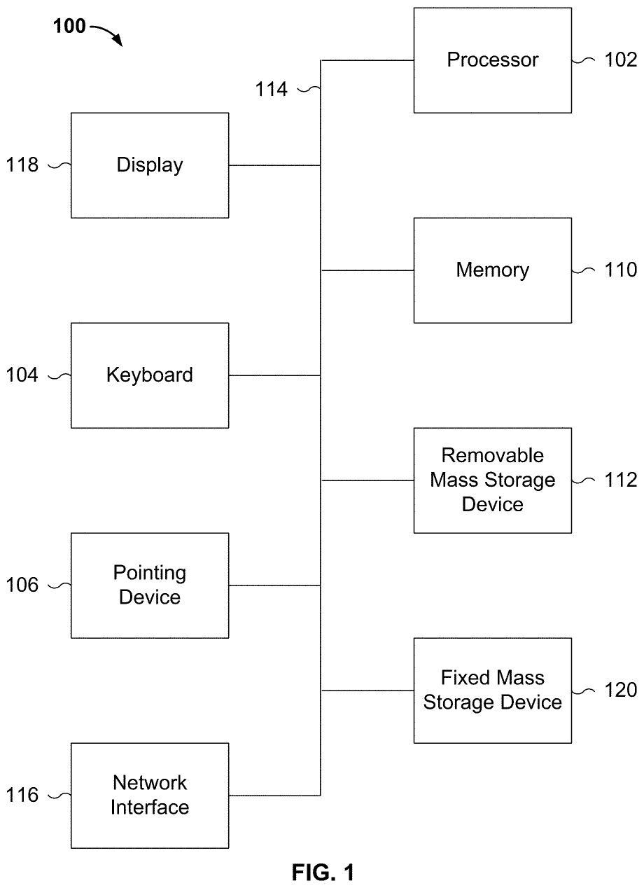

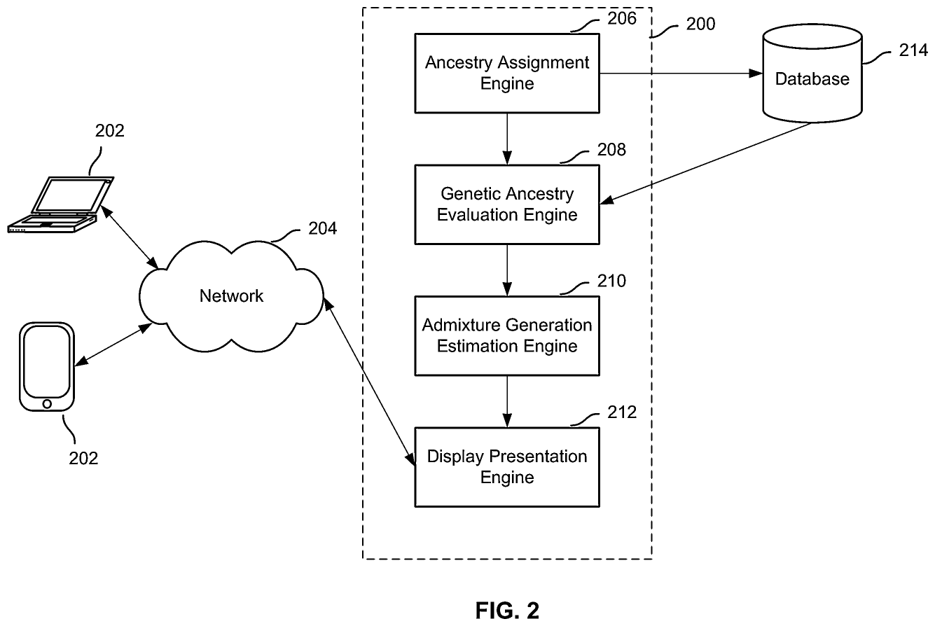

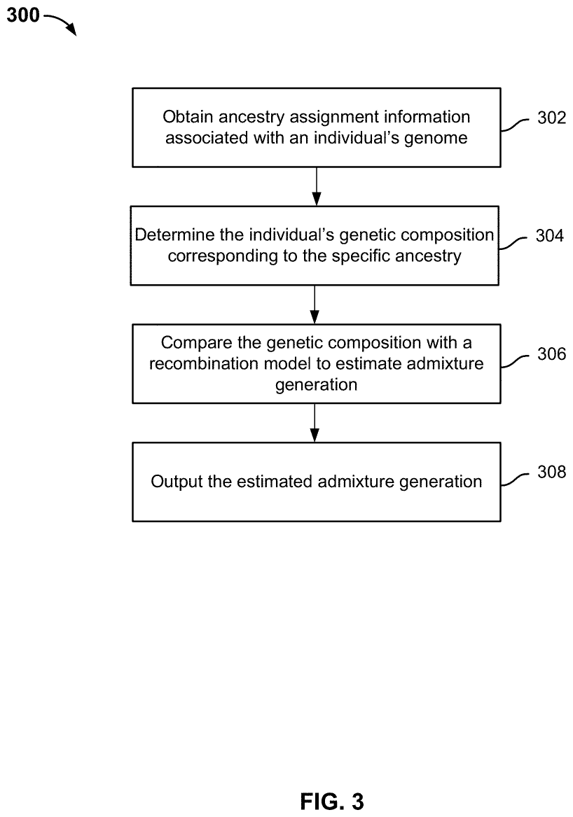

FIG. 1 is a functional diagram illustrating a programmed computer system for admixture generation estimation in accordance with some embodiments. As will be apparent, other computer system architectures and configurations can be used to perform admixture generation estimation. Computer system 100, which includes various subsystems as described below, includes at least one microprocessor subsystem (also referred to as a processor or a central processing unit (CPU)) 102. For example, processor 102 can be implemented by a single-chip processor or by multiple processors. In some embodiments, processor 102 is a general purpose digital processor that controls the operation of the computer system 100. Using instructions retrieved from memory 110, the processor 102 controls the reception and manipulation of input data, and the output and display of data on output devices (e.g., display 118). In some embodiments, processor 102 includes and/or is used to provide engines described below with respect to FIG. 2 and/or executes/performs the processes described below with respect to FIG. 3.

Processor 102 is coupled bi-directionally with memory 110, which can include a first primary storage, typically a random access memory (RAM), and a second primary storage area, typically a read-only memory (ROM). As is well known in the art, primary storage can be used as a general storage area and as scratch-pad memory, and can also be used to store input data and processed data. Primary storage can also store programming instructions and data, in the form of data objects and text objects, in addition to other data and instructions for processes operating on processor 102. Also as is well known in the art, primary storage typically includes basic operating instructions, program code, data and objects used by the processor 102 to perform its functions (e.g., programmed instructions). For example, memory 110 can include any suitable computer-readable storage media, described below, depending on whether, for example, data access needs to be bi-directional or uni-directional. For example, processor 102 can also directly and very rapidly retrieve and store frequently needed data in a cache memory (not shown).

A removable mass storage device 112 provides additional data storage capacity for the computer system 100, and is coupled either bi-directionally (read/write) or uni-directionally (read only) to processor 102. For example, storage 112 can also include computer-readable media such as magnetic tape, flash memory, PC-CARDS, portable mass storage devices, holographic storage devices, and other storage devices. A fixed mass storage 120 can also, for example, provide additional data storage capacity. The most common example of mass storage 120 is a hard disk drive. Mass storages 112, 120 generally store additional programming instructions, data, and the like that typically are not in active use by the processor 102. It will be appreciated that the information retained within mass storages 112 and 120 can be incorporated, if needed, in standard fashion as part of memory 110 (e.g., RAM) as virtual memory.

In addition to providing processor 102 access to storage subsystems, bus 114 can also be used to provide access to other subsystems and devices. As shown, these can include a display monitor 118, a network interface 116, a keyboard 104, and a pointing device 106, as well as an auxiliary input/output device interface, a sound card, speakers, and other subsystems as needed. For example, the pointing device 106 can be a mouse, stylus, track ball, or tablet, and is useful for interacting with a graphical user interface.

The network interface 116 allows processor 102 to be coupled to another computer, computer network, or telecommunications network using a network connection as shown. For example, through the network interface 116, the processor 102 can receive information (e.g., data objects or program instructions) from another network or output information to another network in the course of performing method/process steps. Information, often represented as a sequence of instructions to be executed on a processor, can be received from and outputted to another network. An interface card or similar device and appropriate software implemented by (e.g., executed/performed on) processor 102 can be used to connect the computer system 100 to an external network and transfer data according to standard protocols. For example, various process embodiments disclosed herein can be executed on processor 102, or can be performed across a network such as the Internet, intranet networks, or local area networks, in conjunction with a remote processor that shares a portion of the processing. Additional mass storage devices (not shown) can also be connected to processor 102 through network interface 116.

An auxiliary I/O device interface (not shown) can be used in conjunction with computer system 100. The auxiliary I/O device interface can include general and customized interfaces that allow the processor 102 to send and, more typically, receive data from other devices such as microphones, touch-sensitive displays, transducer card readers, tape readers, voice or handwriting recognizers, biometrics readers, cameras, portable mass storage devices, and other computers.

In addition, various embodiments disclosed herein further relate to computer storage products with a computer readable medium that includes program code for performing various computer-implemented operations. The computer-readable medium is any data storage device that can store data which can thereafter be read by a computer system. Examples of computer-readable media include, but are not limited to, all the media mentioned above: magnetic media such as hard disks, floppy disks, and magnetic tape; optical media such as CD-ROM disks; magneto-optical media such as optical disks; and specially configured hardware devices such as application-specific integrated circuits (ASICs), programmable logic devices (PLDs), and ROM and RAM devices. Examples of program code include both machine code, as produced, for example, by a compiler, or files containing higher level code (e.g., script) that can be executed using an interpreter.

The computer system shown in FIG. 1 is but an example of a computer system suitable for use with the various embodiments disclosed herein. Other computer systems suitable for such use can include additional or fewer subsystems. In addition, bus 114 is illustrative of any interconnection scheme serving to link the subsystems. Other computer architectures having different configurations of subsystems can also be utilized.

FIG. 2 is a block diagram illustrating an embodiment of a system for admixture generation estimation.

In this example, a user uses a client device 202 to communicate with an admixture generation estimation system 200 via a network 204. Examples of device 202 include a laptop computer, a desktop computer, a smart phone, a mobile device, a tablet device, a wearable networking device, or any other appropriate computing device.

Admixture generation estimation system 200 is configured to estimate how many generations ago an individual had an ancestor of a particular ancestry, and present the estimation results for display. Admixture generation estimation system 200 can be implemented on a networked platform (e.g., a server or cloud-based platform, a peer-to-peer platform, etc.) that supports various applications, such as 23andMe®'s personal genome service platform. For example, embodiments of the platform perform admixture generation estimations and provide users with access (e.g., via appropriate user interfaces and communication channels implemented using browser-based applications, standalone applications, etc.) to their personal genetic information (e.g., genetic sequence information and/or genotype information obtained by assaying genetic materials such as blood or saliva samples) and estimated admixture generation information. In some embodiments, the platform also allows users to connect with each other and share information. System 100 can be used to implement 202 or 200.

In some embodiments, genetic samples (e.g., saliva, blood, etc.) are collected from individuals and analyzed using DNA microarray or other appropriate techniques. The individuals' genotype information is obtained (e.g., from genotyping chips directly or from genotyping services that provide assayed results) and stored in database 214. The genotype data can include fully sequenced genome data, Single Nucleotide Polymorphism (SNP) data, exonic data pertaining to exons (the coding portion of genes that are expressed), other assayed DNA marker data (e.g., short tandem repeats (STRs), Copy-Number Variants (CNVs), etc.), as well as any other appropriate form of genetic data pertaining to the individual's genome. In this example, the genotype data is used by system 200 to estimate parental contributions to individuals' ancestries. Results of the estimation can be stored to database 214 or any other appropriate storage unit. Although SNP-based DNA information is discussed for purposes of illustration, the technique is also applicable to other forms of genomic data.

In this example, system 200 includes an ancestry assignment engine 206, a genetic ancestry evaluation engine 208, an admixture generation estimation engine 210, and a display presentation engine 212. In some embodiments, ancestry assignment engine 206 is implemented using an ancestry composition tool such as 23andMe's Geographic Ancestry Analyzer®, which determines the individual's ancestry composition based on the individual's genomic information and generates the ancestry assignments for chromosome segments. Individuals with ancestries from different geographical regions are found to have different genetic variations in certain gene locations. In some embodiments, genome reference models are obtained based on genomes of reference individuals that are known to have specific ancestries. For example, a genome reference model can be obtained based on an un-admixed individual who is known to have four grandparents born in the same geographical region. For example, the Geographic Ancestry Analyzer® employs reference models from geographical regions such as Native America, Northern Europe, Southern Europe, and many other geographical regions or subregions. In some embodiments, segments of an individual's chromosomes are compared with the reference models to find matches and determine the most likely ancestry for each segment accordingly (e.g., if a particular chromosome segment is found to match a corresponding chromosome segment at the same location in the Scandinavian model, then that chromosome segment of the individual user is assigned Scandinavian ancestry). Known techniques for finding chromosome segment matches and assigning ancestries can be used. The ancestry assignment data can be stored in database 214, output to genetic ancestry evaluation engine 208 for further processing, or both.

To determine admixture generation, genetic ancestry evaluation engine 208 obtains ancestry assignment data directly from ancestry assignment engine 206 or from database 214. At least some of the obtained ancestry assignment data indicates that certain segments of an individual's genotype data are deemed to be associated with a specific ancestry. Genetic ancestry evaluation engine 208 determines various genetic ancestry summary data based on ancestry assignment information. The parameters are sent to an admixture generation estimation engine 210, which uses a recombination model and the parameters to estimate the admixture generation. The recombination model is used to generate simulations which are used to compare with summary data, as well as to estimate the admixture generations. Details of the recombination model are described below. The display presentation engine 212 renders and displays the estimation results, or sends the estimation results to be rendered and displayed on a client.

The engines described above can be implemented as software components executing on one or more processors, as hardware such as programmable logic devices and/or Application Specific Integrated Circuits designed to perform certain functions or a combination thereof. In some embodiments, the engines can be embodied by a form of software products which can be stored in a nonvolatile storage medium (such as optical disk, flash storage device, mobile hard disk, etc.), including a number of instructions for making a computer device (such as personal computers, servers, network equipment, etc.) implement the methods described in the embodiments of the present application. The engines may be implemented on a single device or distributed across multiple devices. The functions of the engines may be merged into one another or further split into multiple sub-components.

FIG. 3 is a flowchart illustrating an embodiment of a process for admixture generation estimation.

At 302, ancestry assignment information associated with an individual's genotype data is obtained. The ancestry assignment information indicating one or more portions of the individual's genotype data is deemed to be associated with one or more ancestries. As discussed above, in some embodiments, the ancestry assignment information is determined by comparing the individual's chromosome segments to various reference ancestry models, making probabilistic determinations of the likelihood that specific segments correspond to specific ancestries, and making assignments for each segment if the corresponding likelihood at least meets a certain threshold. Any other appropriate techniques for assigning estimated ancestries to segments of the individual's genome can be used. In some embodiments, the ancestry assignments include specifications of the starting and ending positions of the segments and their assignments (e.g., chromosome 1, position 1-position 15, Scandinavian; chromosome 1, position 16-20, German, etc.). Other data formats can be used. For example, the chromosome identifiers and ancestries can be encoded to reduce memory use (e.g., 1:1-15:S, 1:16-20:G, etc.). In this case, the assignments associated with a specific ancestry (e.g., German, Scandinavian, etc.) are selected for further processing. In various embodiments, the ancestry assignment information can be received from an ancestry evaluation engine (e.g., 23andMe's Geographic Ancestry Analyzer®) or the like, or read from a storage location.

At 304, given a specific ancestry, the individual's genetic ancestry summary data corresponding to the specific ancestry is determined. In some embodiments, the genetic ancestry summary data includes various types of data such as the number of segments corresponding to the specific ancestry, the number of chromosomes carrying these segments, the length of each segment (e.g., in centimorgans or megabases), etc. In some embodiments, the total length of the segments, the mean length of the segments, and/or the longest segment length is also included; alternatively, these summary data can be derived based on the lengths of the individual segments. In some embodiments, the genetic ancestry summary data includes the list of segments corresponding to the specific ancestry, and the other types of data (e.g., segment lengths, mean length, number of segments, etc.) can be derived from the list.

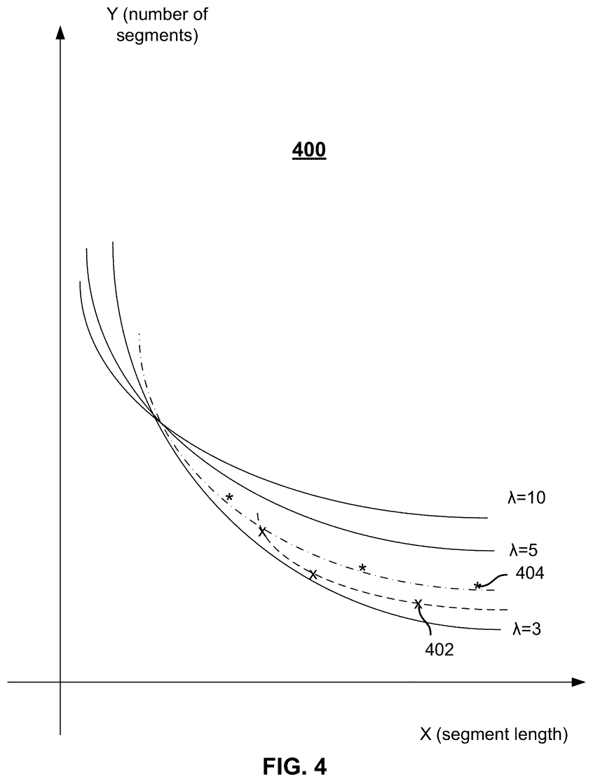

Recombination breaks down segments of a specific ancestry during meiosis, and shortens the segment length. Thus, the shorter the segments of a particular ancestry, the further back in generations the ancestry is traced. On the other hand, the longer the segments of a particular ancestry, the more recent in generations the ancestry is traced. At 306, at least some of the individual's genetic ancestry summary data corresponding to the specific ancestry (also referred to as the observed data) is compared with a recombination model (also referred to as a Poisson model of recombination) to estimate the admixture generation associated with the specific ancestry. In some embodiments, a maximum likelihood determination is made based on the individual's genetic ancestry summary data and the recombination model to determine the most likely admixture generation or range of admixture generations for a full-blooded ancestor of the specific ancestry. Details of the recombination model and the estimation are described below in connection with FIGS. 4-6.

At 308, the estimated admixture generation is output. In some embodiments, the estimated admixture generation is sent to a display and presented to the individual via a user interface.

In some embodiments, a process simulating recombination events that occur when DNAs are admixed is used to generate the recombination model. For example, to simulate four generations of admixing, the chromosomes of eight hypothetical couples are created. In some embodiments, it is assumed that one simulated individual of the sixteen simulated individuals is un-admixed and has full ancestry from the geographical region of interest. The DNAs of each couple are randomly shuffled (subject to known recombination principles) to produce a set of simulated chromosomes for a simulated offspring. The eight simulated offspring are paired and each new couple's DNAs are randomly shuffled again to produce another generation of simulated offspring, and the process is repeated until at the fourth generation a single simulated individual's DNA is generated. The genetic ancestry summary data of this simulated individual's DNA is used to construct a part of the model. In some embodiments, 2-10 generations of admixing are simulated to construct the model. Other ranges can be used. The simulation process is run multiple times for each generation value.

FIG. 4 is a diagram illustrating an example of a recombination model. Model 400 uses the Poisson model of recombination events to generate simulations of admixing over a number of generations. In this example, for purposes of visualization, the genetic ancestry summary data used by the model includes only two factors: variable X corresponds to the length of the segment, and variable Y corresponds to the number of segments in the genome for that length. Including more genetic ancestry summary data factors will result in models with greater numbers of dimensions. In the example shown, each simulated curve is an exponential distribution function based on λ, which corresponds to the number of generations ago the DNA of an ancestor of a particular ancestry became admixed.

If a certain portion (e.g., 1/16) of an individual's DNA segments is from a given ancestry, there are many possibilities for admixture generation: the amount of ancestry can be inherited from two full-blooded ancestors one generation ago, four ancestors two generations ago, eight ancestors three generations ago, etc. When there are more generations, the segments tend to be shorter. In this example, model 400 takes into account the segment lengths and the length of the segments to determine admixture generation for an individual. In the example shown, the number of admixture generations is represented as λ.

In this example, the individual's genetic ancestry summary data includes the lengths of DNA segments assigned for the particular ancestry and the number of segments corresponding to each length. During 306 of process 300, to compare the genomic composition with the recombination model, a maximum likelihood determination is performed using the individual's genomic composition data to identify the curve in the model that most closely resembles the observed data of the individual. As shown in FIG. 4, the observed data set 402 most closely fits the curve that has a λ of 3.

In some cases, the individual's genetic ancestry summary data is consistent with several admixture generation values. Thus, a range of generations is determined. For example, if an individual's genetic ancestry summary data includes data set 404 which is consistent with curves with λ between 3-5, then it is determined that the individual has a full-blooded ancestor of the specific ancestry 3-5 generations ago.

The model shown in FIG. 4 includes two types of genetic ancestry summary data. In some embodiments, additional factors are taken into account to generate a comprehensive model. Table 1 illustrates another example recombination model used to determine the admixture generation. Table 1 maps admixture generations to various types of genetic ancestry summary data, and the values in the table are determined based on simulation results of the recombination simulation process described above. Genetic ancestry summary data obtained from the simulation is recorded for each generation. In this example, the genetic ancestry summary data includes the mean length of chromosome segments corresponding to the ancestry, the length of the longest segment corresponding to the ancestry, the number of chromosome segments corresponding to the ancestry, and the number of chromosomes bearing segments corresponding to the ancestry. Other genetic ancestry summary data can be used in other embodiments. The values and units used are for purposes of illustration only and are not necessarily actual values used. As shown, each entry of the summary data is represented as a range of values obtained based on the statistical distribution of the simulated data. How to select the range depends on implementation. For example, the range for “longest length” can be the range of obtained simulated values 2 standard deviations within the mean longest length value.

TABLE 1

| | | | Number of | Number of |
|---|---|---|---|---|
| | Mean | Longest | segments | chromosomes |
| Number of | length | length | corresponding to | bearing the |
| generations | (ML) | (LL) | the ancestry (NS) | ancestry (NC) |
| 2 | 100+ | 200+ | 4-5 | 18-40 |
| 3 | 11-20 | 50-100 | 5-6 | 15-34 |
| 4 | 10-15 | 40-80 | 10-15 | 12-20 |
| 5 | 8-12 | 35-60 | 20-30 | 10-18 |
| 6 | 5-10 | 22-47 | 16-28 | 8-15 |
| 7 | 3-6 | 16-25 | 9-20 | 5-10 |
| 8 | 2-4 | 9-18 | 7-9 | 4-8 |
| . . . | . . . | . . . | | |

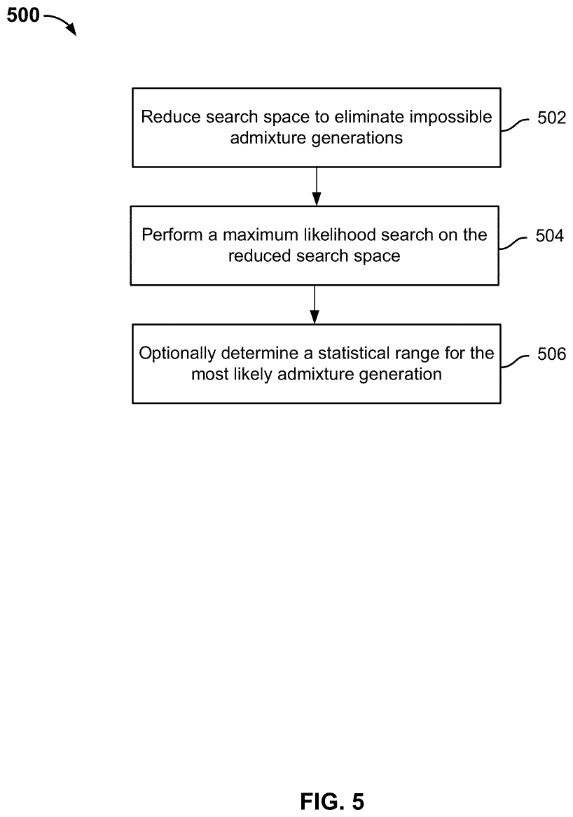

FIG. 5 is a flowchart illustrating an embodiment of a process for estimating admixture generation using a model such as the model represented using Table 1. Process 500 can be used to implement 306 of process 300.

The objective of the admixture generation estimation is to find the most likely admixture generation (or range of admixture generations) that conforms to the individual's genetic ancestry summary data. The full set of data in Table 1 represents the full search space.

In some embodiments, the individual's genetic ancestry summary data can be applied to the model to find in the full search space the most likely admixture generation. Preferably, however, the search space is reduced before the search for the most likely admixture generation or generation range is performed. The reduction is performed because unlike a population-based study where lots of data is available from many individuals, in process 500, there is only one individual's data available to match data in the model. A reduced search space will ensure a more reasonable maximum likelihood search result given the limited amount of data to perform the search. Further, the amount of computation that is required is also reduced as a result of the search space reduction.

Accordingly, at 502, given the individual's genetic ancestry summary data, the search space is reduced to eliminate impossible admixture generations. The following example illustrates the principle of the search space reduction: assume that for a hypothetical individual, there was one full Italian ancestor at the grandparents generation (that is, an admixture generation of 3). The recombination model will determine the possible ways the hypothetical individual inherits the chromosome segments associated with that ancestry. The hypothetical individual can inherit between 12.5%-25% of the Italian ancestry-related chromosome segments from that grandparent. Thus, if an individual has 2% Italian ancestry, the individual's parents or grandparents cannot have full Italian ancestry (in other words, admixture generations 2 and 3 are ruled out).

Now refer to Table 1 for another example. In some embodiments, the individual's genetic ancestry summary data is looked up in the table to find matching ranges and corresponding generations. In such embodiments, the ranges of generations in the model give both the upper bound and the lower bound. Suppose that a user's Italian ancestry summary data has ML, LL, NS, and NC of 10, 45, 18, and 13, respectively, and the corresponding feasible ranges of generations based on ML, LL, NS, and NC ranges of the model are 4-6, 4-6, 6-7, and 4-6, respectively, and the intersection of these ranges gives an overall estimate of 6 generations.

Although the above embodiment is useful for determining the range of feasible generations, it can produce inconsistent results due to imperfections in the model. For example, suppose that an individual's Italian ancestry summary data has ML, LL, NS, and NC values of 10, 44, 9, and 13, and a lookup in the model yields feasible ranges of 4-6, 4-6, 7-8, and 4-6, respectively. Note that the intersection of these ranges is null, indicating that there are inconsistencies in the predicted number of generations. One potential cause of the inconsistency is that the particular model used in this example assumes that there is only one full-blooded ancestor from a specific generation, while in reality the individual can have multiple full-blooded ancestors from one or more generations, which can thus cause the individual's ancestry summary values to be higher than anticipated by the model. In some embodiments, to compensate for this effect during the reduction process, a generation is only ruled out if the individual's data is below the lower bound of the model's range. In other words, for a piece of summary data, the model only provides a lower bound on the generation but not an upper bound. For instance, given that the individual's ML is 10, only generations 2 and 3 are ruled out, while generations 7, 8, and beyond are not ruled out. Although the ML value of 10 is greater than the ML ranges corresponding to these more distant generations (7, 8, and beyond), these generations are still feasible because the individual's higher ML value could be the result of having more than one Italian ancestor from any of these generations. Accordingly, the feasible ranges of generations based on ML, LL, NS, and NC ranges are 4 or more generations, 4 or more generations, 7 or more generations, and 4 or more generations, respectively, giving an intersection/overall range of 7 or more generations.

In some embodiments, the reduction technique is further refined by letting some of the ancestry summary data to set only the lower bounds of the generation ranges but allowing another portion of the ancestry summary data to set both the upper and lower bounds. For example, ML, NS, and NC set the lower bounds but no upper bounds; LL sets the lower bound, but if the measured LL of the individual is greater than 2× the upper bound of the LL range of a generation, that generation and more remote generations are also ruled out. Thus, using the same example where the individual's Italian ancestry summary data has ML, LL, NS, and NC values of 10, 44, 9, and 13, respectively, the generational ranges determined based on ML, NS, and NC are 4 or more generations, 7 or more generations, and 4 or more generations, respectively. The measured LL of 44 is more than 2× the upper bound of the LL range of 8 generations, thus 8 generations and more are ruled out, giving a range of 4-7 generations. The overall intersection is 7 generations.

Returning to FIG. 5, at 504, a maximum likelihood search is performed on the reduced search space, based on the individual's genetic ancestry summary data. In some embodiments, the following likelihood function is used:

L(λ)=Πᵢ₌₁ⁿλ exp(−λxᵢ) (1)

wherein λ corresponds to the number of generations, n corresponds to the number of segments according to the individual's genetic ancestry summary data, and xᵢ corresponds to the length of segment i. Assume that the feasible range is 7-9, then λ of 7, 8, and 9 are tested. L(7), L(8), and L(9) are computed, and the λ that yields the highest value is selected as the most likely admixture generation.

In some embodiments, it is assumed that at the earliest generation, there is only one full-blooded ancestor of that ancestry. Other assumptions can be used for different models or used to augment the existing model. In some embodiments, additional parameters of the individual's chromosomes are optionally determined and used to provide further refinement in estimation. For example, the percentage of chromosome associated with this ancestry (P) (or equivalently, the total length of DNA segments associated with the ancestry), the length of the longest chromosome segment associated with the ancestry (LL), etc.

The additional parameters can be used to further refine the model. For example, in some embodiments, λ′=λ/(1−P) is used, where (1−P) is a correction factor where P is the proportion of the genome that is deemed to be associated with the ancestry. The correction factor corrects for unobserved recombinations, which can occur when multiple full-blooded ancestors at a certain generation contribute to the same ancestry (e.g., two fully Scandinavian great-great-grandparents). In such cases, the recombined segment lengths do not shorten as in the case of a single full-blooded ancestor. The corrected λ′ can be used instead of λ in function (1) for evaluating the likelihoods and selecting a most likely admixture generation.

In some embodiments, after a most likely admixture generation is determined in 504, a statistical range for the most likely admixture generation is optionally determined at 506 to more accurately reflect the statistical variability in the admixture generation determination.

In some embodiments, the statistical range is determined by looking up the statistical range that corresponds to the determined most likely admixture generation in a mapping table such as Table 2.

TABLE 2

| Most likely generation determined | Possible range |
|---|---|
| 2 | 2-3 |
| 3 | 3-4 |
| 4 | 3-5 |
| 5 | 3-7 |
| 6 | 4-9 |
| . . . | . . . |

In some embodiments, Table 2 is generated by applying the admixture estimation process to a reference population with known admixture generations, and mapping the known admixture generations to their respective ranges of estimated results. In particular, the reference population can be a population of real individuals whose admixture generations are known; however, given that ancestry information for remote ancestors is usually unknown, a population of simulated individuals is used in some embodiments. Each simulated individuals is generated using the same recombination simulation process described above, with a single full-blooded ancestor at the i-th generation. Thus, for each simulated individual, the corresponding i is referred to as the truth data. Each simulated individual's genetic ancestry summary data is evaluated, and 502 of process 500 is performed to reduce the search space and determine the range of possible admixture generations. 504 of process 500 is also performed to determine the most likely admixture generation. Specifically, function (1) is applied to each possible admixture generation λ to determine the corresponding value of L(λ), and the admixture generation that gives the highest L(λ) is selected as the most likely admixture generation. Different simulated individuals with the same admixture generation value i (that is, the same truth data) can lead to different most likely admixture generation results because they inherit different amounts and lengths of chromosomes from a full-blooded ancestor. For example, suppose that simulated individuals with truth data i=3, i=4, i=5, and i=6 can lead to estimated most likely ranges of 3-5, 3-6, 4-7, and 5-9, respectively. Thus, for an estimated most likely admixture generation value of 4, based on the likely range to truth data mapping above, the possible range of truth data is i=3-5. Entries in Table 2 are thus constructed to give insight into given a determined most likely range, what is actually the possible range of truth data. Although a table is used for purposes of illustration, other appropriate forms such as a function, a list, etc., can be used.

Once the admixture generation is determined, the display engine presents the information to be displayed (e.g., sent over the network to be displayed on a client device, or displayed directly if a client application is executing on the admixture generation estimation system).

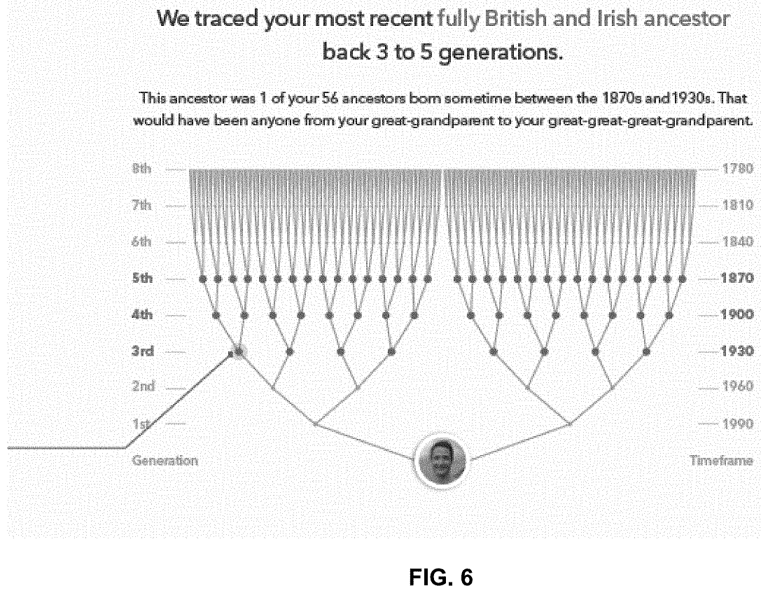

FIG. 6 is a user interface diagram illustrating an example screen displaying admixture generation information. In this example, an individual is found to have at least one full-blooded ancestor from the geographical area of Britain and Ireland between three to five generations ago. Thus, an ancestry tree is displayed to the individual (the user), with each node on the tree corresponding to an ancestor of a specific generation, and each row of nodes corresponding to a specific generation. The estimated birth dates of the ancestors are determined (e.g., each generation of parents is estimated to be born thirty years before the child) and displayed.

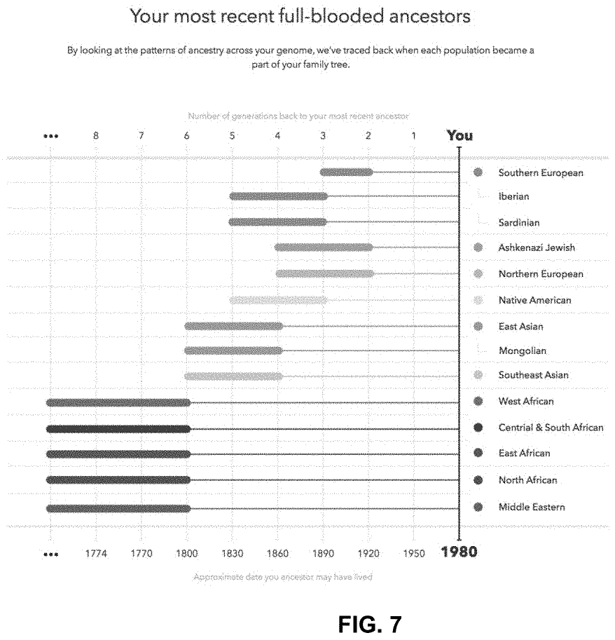

FIG. 7 is a user interface diagram illustrating another example screen displaying admixture generation information. In this example, the individual is determined to have multiple genetic ancestries. These genetic ancestries and the corresponding estimated admixed generations (in this case, a range of possible generations) are displayed to the individual (the user).

Displays such as FIGS. 6 and 7 help an individual user, who is typically not a genetics expert, better comprehend his/her genetic ancestries and more easily learn about the admixing of his/her ancestors.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A computer-implemented method, comprising:
comparing, by way of one or more processors, representations of a plurality of genetic segments of an individual to a plurality of reference ancestry models, wherein the reference ancestry models are stored in a database and are respectively associated with different ancestries;
determining, by way of the one or more processors and based on the comparison, that one or more genetic segments of the plurality of genetic segments correspond to a specific ancestry;
determining, by way of the one or more processors and for the individual, genetic ancestry summary data corresponding to the specific ancestry, wherein the genetic ancestry summary data includes at least one of: a number of the one or more genetic segments that correspond to the specific ancestry, a number of chromosomes carrying the one or more genetic segments that correspond to the specific ancestry, or lengths of each of the one or more genetic segments that correspond to the specific ancestry;
applying, by way of the one or more processors, maximum likelihood estimation to the genetic ancestry summary data as fit to a genetic recombination model, the genetic recombination model associating respective genetic characteristics to predicted numbers of generations between a subject having genetic characteristics and at least one non-admixed ancestor of the specific ancestry;
determining, by way of the one or more processors and from the maximum likelihood estimation, one or more estimated numbers of generations between the individual and a non-admixed ancestor of the individual, the non-admixed ancestor having the specific ancestry; and
providing, by way of the one or more processors and for graphical display on a user interface, the one or more estimated numbers of generations and the specific ancestry.

2. The computer-implemented method of claim 1, wherein the genetic ancestry summary data also includes at least one of: a list of the one or more genetic segments that correspond to the specific ancestry, a mean length of the one or more genetic segments that correspond to the specific ancestry, or a longest segment length of the one or more genetic segments that correspond to the specific ancestry.

3. The computer-implemented method of claim 1, wherein the genetic recombination model is generated based at least in part on a simulation of recombination events for a plurality of generations of admixing.

4. The computer-implemented method of claim 1, wherein the genetic recombination model is based at least in part on exponential curves representing lengths of the one or more genetic segments versus numbers of the one or more genetic segments of each of the lengths, and wherein the maximum likelihood estimation fits the genetic ancestry summary data to one or more of the exponential curves.

5. The computer-implemented method of claim 1, wherein the genetic recombination model is based at least in part on a mapping of generations to one or more of: a mean length of the one or more genetic segments that correspond to the specific ancestry, a longest segment length of the one or more genetic segments that correspond to the specific ancestry, the number of the one or more genetic segments that correspond to the specific ancestry, or the number of chromosomes carrying the one or more genetic segments that correspond to the specific ancestry, and wherein the maximum likelihood estimation fits the genetic ancestry summary data to the mapping.

6. The computer-implemented method of claim 5, wherein a search space of the mapping is reduced by eliminating impossible admixture generations prior to fitting the genetic ancestry summary data to the mapping, and wherein the impossible admixture generations are identified based on a percentage of the specific ancestry assigned to the individual.

7. The computer-implemented method of claim 5, wherein the maximum likelihood estimation fits the genetic ancestry summary data to the mapping based on an intersection of feasible ranges of the generations that were determined from data related to the one or more genetic segments.

8. The computer-implemented method of claim 7, wherein the intersection of the feasible ranges is used to determine a lower bound on the generations.

9. The computer-implemented method of claim 1, wherein the one or more estimated numbers of generations include a range of generations.

10. The computer-implemented method of claim 1, wherein the non-admixed ancestor of the individual has been determined to be a full-blooded ancestor of the specific ancestry.

11. The computer-implemented method of claim 1, wherein the specific ancestry is associated with a particular geographic region.

12. A non-transitory computer-readable medium storing program instructions that, when executed by one or more processors of a computing system, cause the computing system to perform operations comprising:
comparing representations of a plurality of genetic segments of an individual to a plurality of reference ancestry models, wherein the reference ancestry models are stored in a database and are respectively associated with different ancestries;
determining, based on the comparison, that one or more genetic segments of the plurality of genetic segments correspond to a specific ancestry;
determining, for the individual, genetic ancestry summary data corresponding to the specific ancestry, wherein the genetic ancestry summary data includes at least one of: a number of the one or more genetic segments that correspond to the specific ancestry, a number of chromosomes carrying the one or more genetic segments that correspond to the specific ancestry, or lengths of each of the one or more genetic segments that correspond to the specific ancestry;
applying maximum likelihood estimation to the genetic ancestry summary data as fit to a genetic recombination model, the genetic recombination model associating respective genetic characteristics to predicted numbers of generations between a subject having genetic characteristics and at least one non-admixed ancestor of the specific ancestry;
determining, from the maximum likelihood estimation, one or more estimated numbers of generations between the individual and a non-admixed ancestor of the individual, the non-admixed ancestor having the specific ancestry; and
providing, for graphical display on a user interface, the one or more estimated numbers of generations and the specific ancestry.

13. The non-transitory computer-readable medium of claim 12, wherein the genetic recombination model is generated based at least in part on a simulation of recombination events for a plurality of generations of admixing.

14. The non-transitory computer-readable medium of claim 12, wherein the genetic recombination model is based at least in part on exponential curves representing lengths of the one or more genetic segments versus numbers of the one or more genetic segments of each of the lengths, and wherein the maximum likelihood estimation fits the genetic ancestry summary data to one or more of the exponential curves.

15. The non-transitory computer-readable medium of claim 12, wherein the genetic recombination model is based at least in part on a mapping of generations to one or more of: a mean length of the one or more genetic segments that correspond to the specific ancestry, a longest segment length of the one or more genetic segments that correspond to the specific ancestry, the number of the one or more genetic segments that correspond to the specific ancestry, or the number of chromosomes carrying the one or more genetic segments that correspond to the specific ancestry, and wherein the maximum likelihood estimation fits the genetic ancestry summary data to the mapping.

16. The non-transitory computer-readable medium of claim 15, wherein a search space of the mapping is reduced by eliminating impossible admixture generations prior to fitting the genetic ancestry summary data to the mapping, and wherein the impossible admixture generations are identified based on a percentage of the specific ancestry assigned to the individual.

17. The non-transitory computer-readable medium of claim 15, wherein the maximum likelihood estimation fits the genetic ancestry summary data to the mapping based on an intersection of feasible ranges of the generations that were determined from data related to the one or more genetic segments.

18. The non-transitory computer-readable medium of claim 17, wherein the intersection of the feasible ranges is used to determine a lower bound on the generations.

19. The non-transitory computer-readable medium of claim 12, wherein the one or more estimated numbers of generations include a range of generations.

20. A computing system comprising:
a processor;
memory; and
program instructions, stored in the memory, that upon execution by the processor cause the computing system to perform operations comprising:
comparing representations of a plurality of genetic segments of an individual to a plurality of reference ancestry models, wherein the reference ancestry models are stored in a database and are respectively associated with different ancestries;
determining, based on the comparison, that one or more genetic segments of the plurality of genetic segments correspond to a specific ancestry;
determining, for the individual, genetic ancestry summary data corresponding to the specific ancestry, wherein the genetic ancestry summary data includes at least one of:
a number of the one or more genetic segments that correspond to the specific ancestry, a number of chromosomes carrying the one or more genetic segments that correspond to the specific ancestry, or lengths of each of the one or more genetic segments that correspond to the specific ancestry;
applying maximum likelihood estimation to the genetic ancestry summary data as fit to a genetic recombination model, the genetic recombination model associating respective genetic characteristics to predicted numbers of generations between a subject having genetic characteristics and at least one non-admixed ancestor of the specific ancestry;
determining, from the maximum likelihood estimation, one or more estimated numbers of generations between the individual and a non-admixed ancestor of the individual, the non-admixed ancestor having the specific ancestry; and
providing, for graphical display on a user interface, the one or more estimated numbers of generations and the specific ancestry.
